(19) United States
(12) Patent Application Publication
(10) Pub. No.: US 2017/0113029 A1
(43) Pub. Date: Apr. 27, 2017

(54) MEDICATION APPLICATION ASSEMBLY

(71) Applicant: Hector Hernandez, Tracy, CA (US)

(72) Inventor: Hector Hernandez, Tracy, CA (US)

(21) Appl. No.: 14/921,386

(22) Filed: Oct. 23, 2015

(51) Int. Cl. A61M 35/00

(52) U.S. Cl. CPC A61M 35/003

(57) ABSTRACT

A medication application assembly includes a rod is elongated and has a first end and a second end. A cylinder has a pair of ends each comprising a spindle. A mount is attached to and extends away from the second end. The mount engages the cylinder such that the cylinder is rotatable with respect to the mount. A sleeve is impregnated with a fluid and is removably positioned on the cylinder.

### BACKGROUND OF THE DISCLOSURE

### Field of the Disclosure

The disclosure relates to medication applying devices and more particularly pertains to a new medication applying device for facilitating self-application of fluids to difficult to reach areas of the body.

SUMMARY OF THE DISCLOSURE

An embodiment of the disclosure meets the needs presented above by generally comprising a rod is elongated and has a first end and a second end. A cylinder has a pair of ends each comprising a spindle. A mount is attached to and extends away from the second end. The mount engages the cylinder such that the cylinder is rotatable with respect to the mount. A sleeve is impregnated with a fluid and is removably positioned on the cylinder.

There has thus been outlined, rather broadly, the more important features of the disclosure in order that the detailed description thereof that follows may be better understood, and in order that the present contribution to the art may be better appreciated. There are additional features of the disclosure that will be described hereinafter and which will form the subject matter of the claims appended hereto.

The objects of the disclosure, along with the various features of novelty which characterize the disclosure, are pointed out with particularity in the claims annexed to and forming a part of this disclosure.

### BRIEF DESCRIPTION OF THE DRAWINGS

The disclosure will be better understood and objects other than those set forth above will become apparent when consideration is given to the following detailed description thereof. Such description makes reference to the annexed drawings wherein:

FIG. 1 is a front perspective view of a medication application assembly according to an embodiment of the disclosure.

FIG. 2 is an exploded perspective view of an embodiment of the disclosure.

FIG. 3 is a side view of an embodiment of the disclosure.

FIG. 4 is a front view of an embodiment of the disclosure.

FIG. 5 is a perspective view of an embodiment of the disclosure.

FIG. 6 is a front exploded view of an embodiment of the disclosure.

### DESCRIPTION OF THE PREFERRED EMBODIMENT

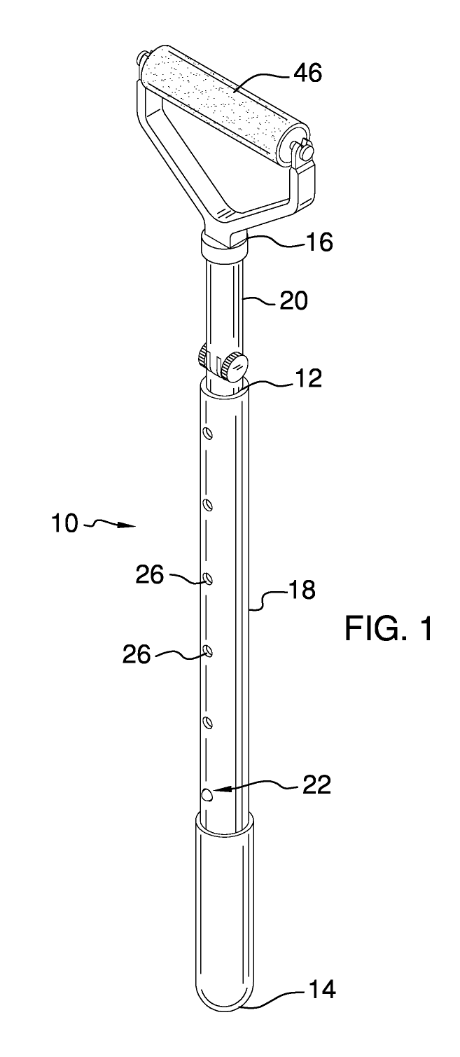

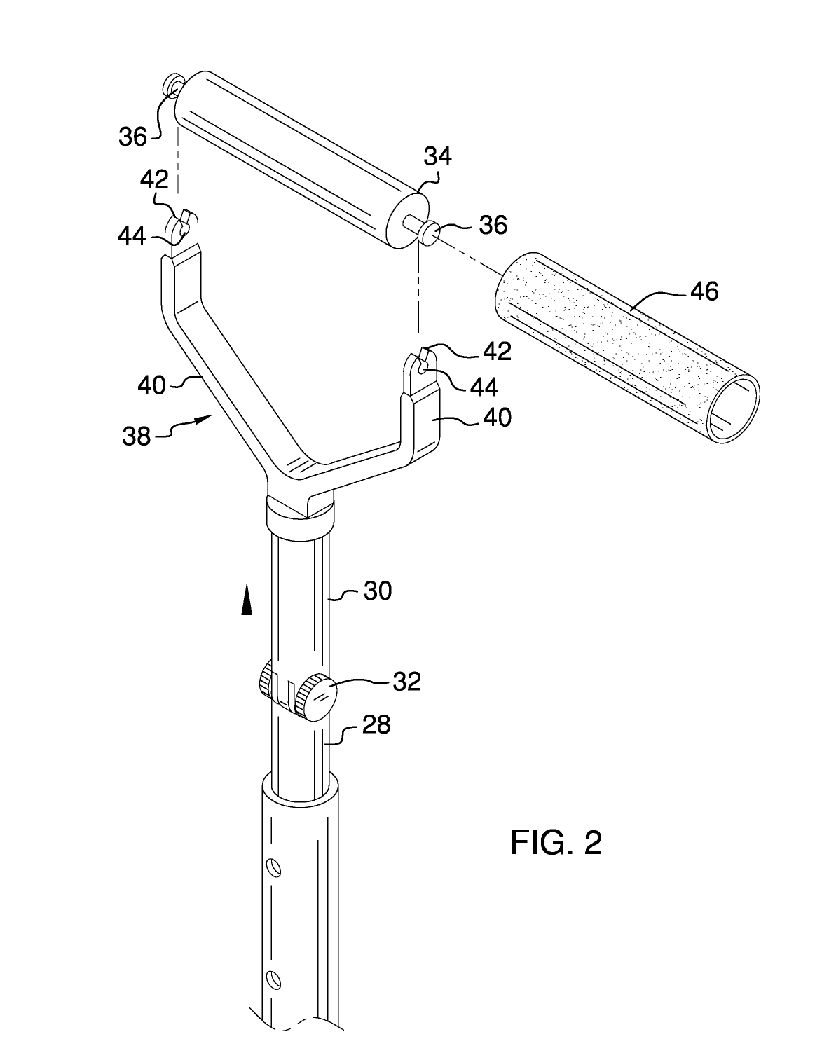

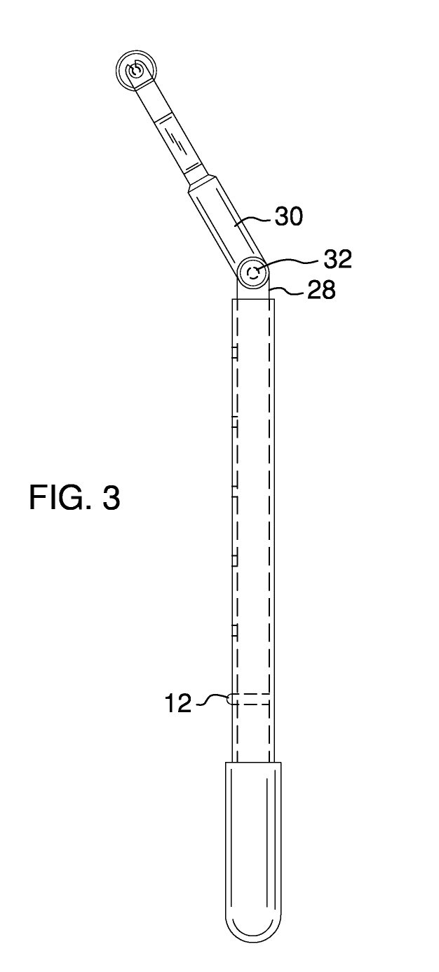

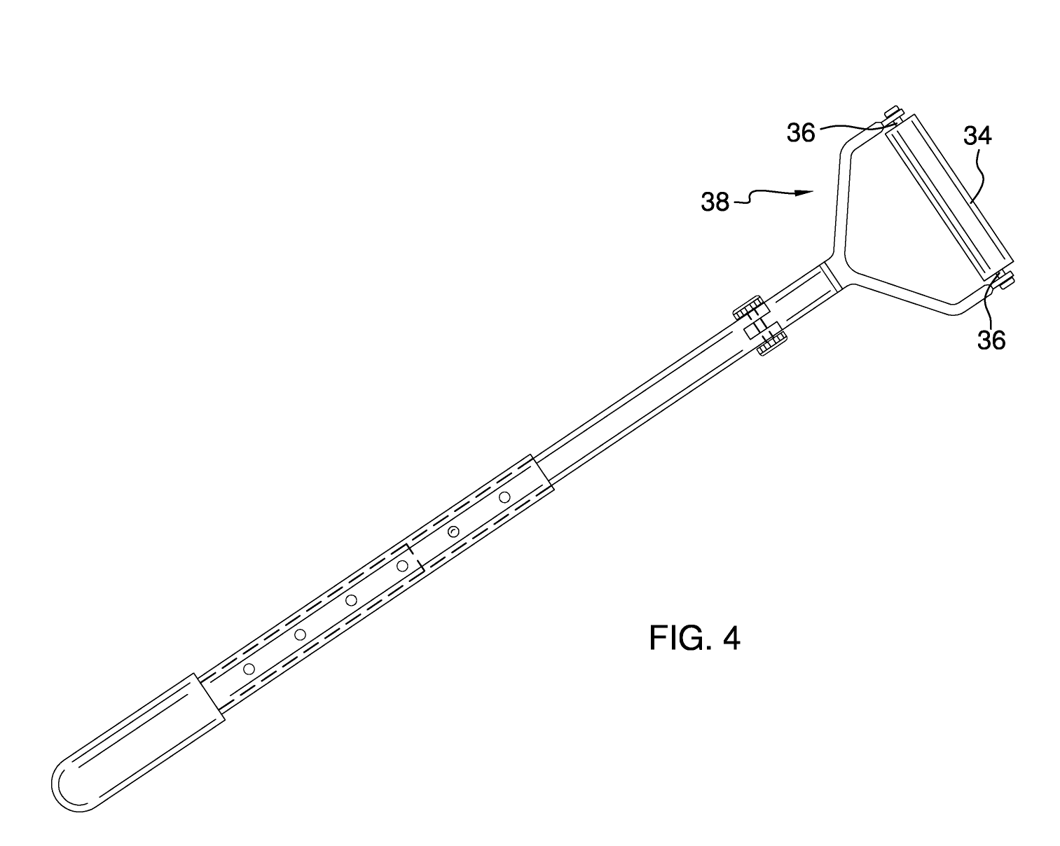

With reference now to the drawings, and in particular to FIGS. 1 through 6 thereof, a new medication applying device embodying the principles and concepts of an embodiment of the disclosure and generally designated by the reference numeral 10 will be described.

As best illustrated in FIGS. 1 through 6, the medication application assembly 10 generally comprises a rod 12 that is elongated and has a first end 14 and a second end 16. The rod 12 is telescopic and has an adjustable length. The rod 12 includes a first member 18 and a second member 20 slidably coupled to each other. A locking member 22 releasably secures the first 18 and second 20 members together such that the length of the rod 12 is fixed. The locking member 22 may comprise a conventional detent 24 extending from the second member 20 and which is positionable in one of a plurality openings 26 of the first member 18. The second member 20 may include a first section 28 and a second section 30 that are pivotally coupled together. A tightening member 32 releasably secures the first 28 and second 30 sections together at a selected angled with respect to each other. The second section 30 includes the second end 16 and the first end 14 may have a grip positioned thereon. The rod 12 has an adjustable length between 8.0 inches and 36.0 inches.

A cylinder 34 has a pair of ends each comprising a spindle 36. A mount 38 is attached to and extends away from the second end 14. The mount 38 is configured to engage the cylinder 34 such that the cylinder 34 is rotatable with respect to the mount 38 and has a rotational axis oriented perpendicular to a longitudinal axis of the rod 12. The mount 38 includes a pair of arms 40 each attached to the second end 16. The arms 40 are angled away from each other. The arms 40 each have a terminal end 42 wherein the terminal ends 42 of the arms 40 are spaced from each other. Each of the terminal ends 42 has a notch 44 therein. The notches 44 each releasably receive one of the spindles 36. The spindles 36 rotate relative to the arms 40.

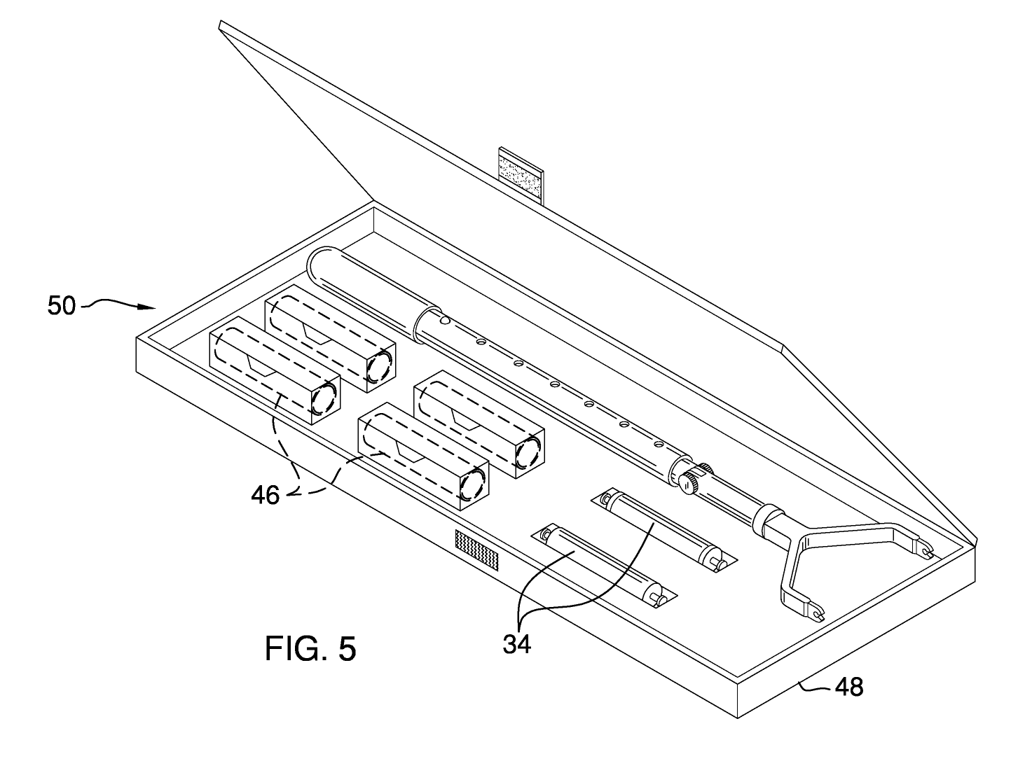

A sleeve 46 is impregnated with a fluid and is removably positioned on the cylinder 34. The sleeve 46 may be comprised of an absorbent material. The fluid 46 may be a medication, lotion or the like which requires absorption by a person's skin. The sleeve 46 and cylinder 34 may be provided as plurality of sleeves 46 and cylinders 34 for interchanging of each and the fluids thereon. FIG. 5 depicts a kit 50 including a case 48 for holding the rod 12 and mount 38 as well as a plurality of cylinders 34 and sleeves 46 wherein the sleeves 46 may be individually packaged. The kit 50 may include additional features such as additional attachments which may be used to replace the second member 20. The additional attachments may include body cleaning implements, back scratchers and the like.

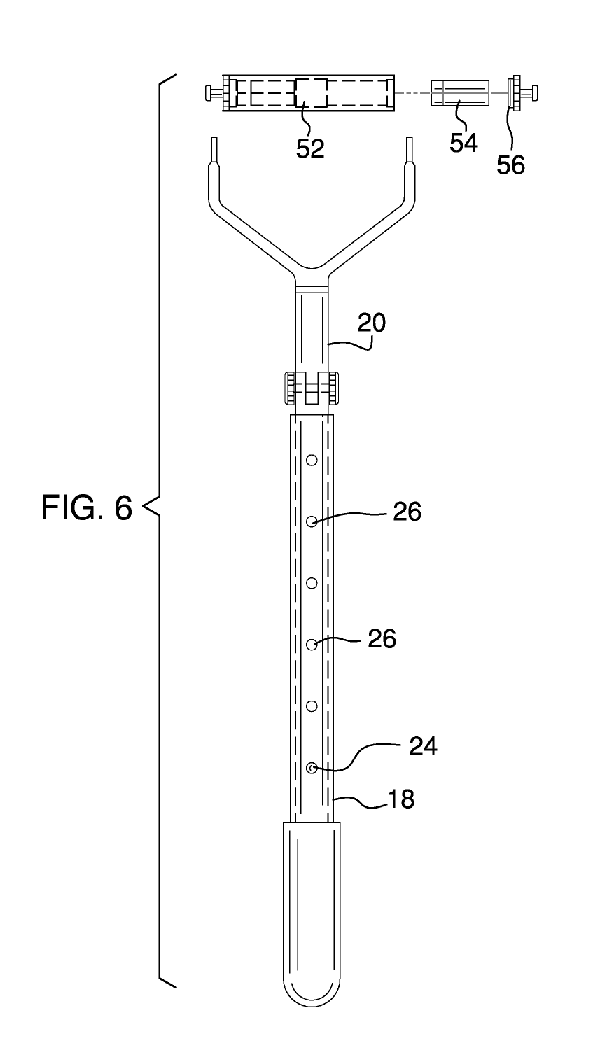

FIG. 6 includes an embodiment having a vibratory motor 52 positioned within the cylinder 34 and powered by one or more batteries 54. The vibratory motor 52 vibrates the cylinder 34 for a massaging and fluid spreading effect. As can be understood by FIG. 6, the cylinder 34 may include a cap 56 for accessing an interior of the cylinder 34 for removal an insertion of batteries 54 into the cylinder 34.

In use, the mount 38 is fitted with a cylinder 34 and sleeve 46 with the appropriate fluid which may include various medications, lotions and the like. The rod 12 is extended as needed to allow the user to more easily reach various portions of their body which would otherwise not be easily accessible. The first 28 and second 30 sections may also be angled to further aid the use of the assembly 10. Additionally, the assembly 10 provides for allowing hands free application of fluids to the body.

With respect to the above description then, it is to be realized that the optimum dimensional relationships for the parts of an embodiment enabled by the disclosure, to include variations in size, materials, shape, form, function and manner of operation, assembly and use, are deemed readily apparent and obvious to one skilled in the art, and all equivalent relationships to those illustrated in the drawings and described in the specification are intended to be encompassed by an embodiment of the disclosure.

Therefore, the foregoing is considered as illustrative only of the principles of the disclosure. Further, since numerous modifications and changes will readily occur to those skilled in the art, it is not desired to limit the disclosure to the exact construction and operation shown and described, and accordingly, all suitable modifications and equivalents may be resorted to, falling within the scope of the disclosure. In this patent document, the word “comprising” is used in its non-limiting sense to mean that items following the word are included, but items not specifically mentioned are not excluded. A reference to an element by the indefinite article “a” does not exclude the possibility that more than one of the element is present, unless the context clearly requires that there be only one of the elements.

I claim:

1. A medication application assembly configured to facilitate spreading of a viscous fluid on a person, said assembly comprising:
a rod being elongated and having a first end and a second end;
a cylinder having a pair of ends each comprising a spindle;
a mount being attached to and extending away from said second end, said mount being configured to engage said cylinder such that said cylinder is rotatable with respect to said mount; and
a sleeve being impregnated with a fluid, said sleeve being removably positioned on said cylinder.

2. The medication application assembly according to claim 1, wherein said rod is telescopic and has an adjustable length.

3. The medication application assembly according to claim 2, wherein said rod includes a first member and a second member slidably coupled to each other, a locking member releasably securing said first and second members together such that the length of said rod is fixed.

4. The medication application assembly according to claim 3, wherein said second member includes a first section and a second section being pivotally coupled together, a tightening member releasably securing said first and second sections together at a selected angled with respect to each other, said second section including said second end.

5. The medication application assembly according to claim 2, wherein said rod has an adjustable length between 8.0 inches and 36.0 inches.

6. The medication application assembly according to claim 1, wherein said mount includes a pair of arms being attached to said second end, said arms each having a terminal end wherein said terminal ends of said arms are spaced from each other, each of said terminal ends having a notch therein, each said notch releasably receiving one of said spindles.

7. A medication application assembly configured to facilitate spreading of a viscous fluid on a person, said assembly comprising:
a rod being elongated and having a first end and a second end, said rod being telescopic and having an adjustable length, said rod including a first member and a second member slidably coupled to each other, a locking member releasably securing said first and second members together such that the length of said rod is fixed, said second member including a first section and a second section being pivotally coupled together, a tightening member releasably securing said first and second sections together at a selected angled with respect to each other, said second section including said second end, said rod having an adjustable length between 8.0 inches and 36.0 inches;
a cylinder having a pair of ends each comprising a spindle;
a mount being attached to and extending away from said second end, said mount being configured to engage said cylinder such that said cylinder is rotatable with respect to said mount, said mount including a pair of arms being attached to said second end, said arms each having a terminal end wherein said terminal ends of said arms are spaced from each other, each of said terminal ends having a notch therein, each said notch releasably receiving one of said spindles; and
a sleeve being impregnated with a fluid, said sleeve being removably positioned on said cylinder.
